(19) United States
(12) Patent Application Publication
(10) Pub. No.: US 2020/0199097 A1
(43) Pub. Date: Jun. 25, 2020

(54) POMALIDOMIDE DERIVATIVE AND PREPARATION METHOD THEREFOR

(71) Applicant: NANJING NORATECH PARMACEUTICALS CO., LTD, Nanjing City, Jiangsu Province (CN)

(72) Inventors: Fei LIU, Nanjing (CN); Gang WU, Nanjing (CN); Xin ZHAO, Nanjing (CN); Pan CHEN, Nanjing (CN); Yunpeng LIAO, Nanjing (CN); Xiaobo WANG, Nanjing (CN); Zhi QI, Nanjing (CN); Xudong YANG, Nanjing (CN)

(73) Assignee: NANJING NORATECH PHARMACEUTICALS CO., LTD, Nanjing City, Jiangsu Province (CN)

(21) Appl. No.: 16/637,938

(22) PCT Filed: Aug. 21, 2017
(86) PCT No.: PCT/CN2017/098281

§ 371 Date: Feb. 10, 2020

(51) Int. Cl. C07D 401/04; C07D 401/14; C07D 405/14; C07F 9/6558

(52) U.S. Cl. CPC C07D 401/04; C07F 9/65583; C07D 405/14; C07D 401/14

(57) ABSTRACT

Disclosed in the present invention are a Pomalidomide derivative and a preparation method therefor. Specifically, the present invention relates to the Pomalidomide derivative and a stereoisomer thereof, or a pharmaceutically acceptable salt, and applications thereof in the preparation of drugs for treating cancers.

### TECHNOLOGY FIELD

The present invention relates to a pomalidomide derivative or a pharmaceutically acceptable salt thereof, and the use thereof in preparing a medicament for treating cancer.

### BACKGROUND OF THE INVENTION

Pomalidomide (formula A) was developed by the American Celgene Corporation and was first approved for marketing in the United States in February 2013. Pomalidomide is the third immunomodulator of the same kind listed after thalidomide and lenalidomide, and can enhance the immune response mediated by T-cells and natural killer cells, while also inhibiting the production of pro-inflammatory cytokines (such as TNF-α, IL-6, etc.) by monocytes. In addition, pomalidomide is capable of inhibiting tumor cell proliferation and inducing apoptosis, and also has a strong inhibitory effect on the proliferation of lenalidomide-resistant multiple myeloma cell lines.

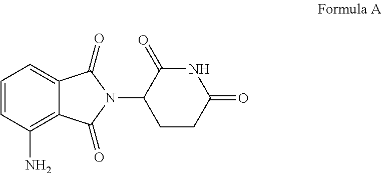 [SMILES: Nc1cccc2c1C(=O)N(C1CCC(=O)NC1=O)C2=O]

Common adverse reactions of pomalidomide include neutropenia, fatigue, weakness, anemia, constipation, diarrhea, thrombocytopenia, upper respiratory tract infections, back pain, and fevers. It may also cause thrombosis, and may cause serious birth defects in fetuses.

According to the literature, it is reported that pomalidomide is a poorly soluble drug, and its solubility when tested in purified water, a phosphate buffer (pH 6.8), an acetate buffer (pH 4.5) and 0.1 mol/L hydrochloric acid is 17.8 μg/mL, 17.0 μg/mL, 18.7 μg/mL and 18.9 μg/mL, respectively. The low solubility of pomalidomide not only increases the difficulty of the preparation process, but also limits the dissolution and absorption process of active ingredients in the gastrointestinal tract, thereby affecting the oral bioavailability.

The pomalidomide oral capsules currently on the market are under the trade name POMALYST, and in order to increase the dissolution of the drug, a surfactant, i.e. sodium lauryl sulfate, is added to the formula. However, the sodium lauryl sulfate may cause a certain amount of irritation for the gastrointestinal tract.

### SUMMARY OF THE INVENTION

An objective of the present invention is to provide a novel pomalidomide prodrug having a high stability, good solubility, improved bioavailability, less toxic side effects or long-acting potential.

In one aspect, the present invention discloses a compound of formula (I) (including a stereoisomer thereof) or a pharmaceutically acceptable salt thereof,

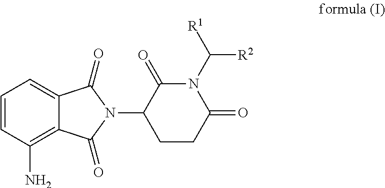

wherein:

R¹ is selected from H, substituted or unsubstituted C₁₋₆ alkyl, substituted or unsubstituted C₁₋₆ alkoxy, substituted or unsubstituted C₂₋₆ alkenyl, or substituted or unsubstituted C₂₋₆ alkynyl, wherein the aforementioned substituted substituent is selected from C₁₋₆ alkyl and C₁₋₆ alkoxy;

R² is selected from H, —OR³, —SR³, —NHR³, substituted or unsubstituted C₃₋₁₀ heterocyclyl, substituted or unsubstituted C₃₋₁₀ heterocyclic aryl, or substituted or unsubstituted C₃₋₁₀ cycloalkyl, wherein the aforementioned substituted substituent is selected from C₁₋₆ alkyl, C₁₋₆ alkoxy, carbonyl, carboxyl, amino, or hydroxy;

R³ is selected from —C(O)(CH)(R⁴)(R⁵), —P(O)(OR⁶)(OR⁷), —P(O)₂(OR⁶)M, or —P(O)₃MY;

R⁴ is selected from hydrogen, amino, hydroxy, halogen, or C₁₋₆ alkyl;

R⁵ is selected from hydrogen, substituted or unsubstituted C₁₋₁₆ alkyl, substituted or unsubstituted C₁₋₆ alkoxy, substituted or unsubstituted C₃₋₁₀ heterocyclyl, substituted or unsubstituted C₃₋₁₀ heterocyclic aryl, substituted or unsubstituted C₃₋₁₀ cycloalkyl, phenyl, benzyl, —(CH₂)nSCH₃, —(CH₂)mNHCH₃, or —(CH₂)mN(CH₃)₂, wherein the aforementioned substituted substituent is selected from amino, hydroxyl, carboxyl, —SH, —C(O)NH₂, C₁₋₆ alkyl;

R⁶ and R⁷ are each independently selected from hydrogen or C₁₋₆ alkyl; M and Y are each independently selected from a monovalent cation, or MY is a divalent cation; and

m and n are each independently selected from 1, 2, 3, 4, 5 or 6.

In one embodiment, R¹ is selected from H, substituted or unsubstituted methyl, substituted or unsubstituted ethyl, substituted or unsubstituted propyl, substituted or unsubstituted butyl, substituted or unsubstituted pentyl, or substituted or unsubstituted hexyl, wherein the aforementioned substituted substituent is selected from methyl, ethyl, or propyl.

In one embodiment, R² is selected from H, —OR³, substituted or unsubstituted tetrahydrofuranyl, substituted or unsubstituted tetrahydropyrrolyl, substituted or unsubstituted furanyl, substituted or unsubstituted thienyl, substituted or unsubstituted pyrrolyl, substituted or unsubstituted imidazolyl, substituted or unsubstituted pyrazolyl, substituted or unsubstituted thiazolyl, substituted or unsubstituted oxazolyl, substituted or unsubstituted pyridyl, substituted or unsubstituted pyrimidyl, substituted or unsubstituted pyridazinyl, substituted or unsubstituted pyrazinyl, substituted or unsubstituted purinyl, substituted or unsubstituted quinolyl, substituted or unsubstituted isoquinolyl, substituted or unsubstituted indolyl, substituted or unsubstituted cyclopropyl, substituted or unsubstituted cyclohexyl, substituted or unsubstituted cyclobutyl, substituted or unsubstituted cyclopentyl,

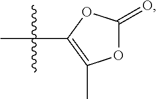 [SMILES: *c1oc(=O)oc1C]

wherein the aforementioned substituted substituent is selected from methyl, ethyl, propyl, carbonyl, carboxyl, amino, or hydroxy.

In a preferred embodiment, R³ is selected from —C(O)(CH)(R⁴)(R⁵), wherein R⁴ is selected from hydrogen, amino, methyl, ethyl, or propyl; R5 is selected from hydrogen, substituted or unsubstituted tetrahydrofuranyl, substituted or unsubstituted tetrahydropyrrolyl, substituted or unsubstituted furanyl, substituted or unsubstituted thienyl, substituted or unsubstituted pyrrolyl, substituted or unsubstituted imidazolyl, substituted or unsubstituted pyrazolyl, substituted or unsubstituted thiazolyl, substituted or unsubstituted oxazolyl, substituted or unsubstituted pyridyl, substituted or unsubstituted pyrimidyl, substituted or unsubstituted pyridazinyl, substituted or unsubstituted pyrazinyl, substituted or unsubstituted purinyl, substituted or unsubstituted quinolyl, substituted or unsubstituted isoquinolyl, substituted or unsubstituted indolyl, substituted or unsubstituted cyclopropyl, substituted or unsubstituted cyclohexyl, substituted or unsubstituted cyclobutyl, substituted or unsubstituted cyclopentyl, phenyl, benzyl, —(CH₂)nSCH₃, —(CH₂)mNHCH₃,—(CH₂)mN(CH₃)₂,

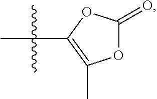 [SMILES: *c1oc(=O)oc1C]

wherein the aforementioned substituted substituent is selected from amino, hydroxy, carboxyl, —SH, —C(O)NH₂, methyl, ethyl, or propyl.

In a more preferred embodiment, n is selected from 1, 2 or 3, and m is selected from 1, 2, 3, 4 or 5.

In one embodiment, R³ is selected from —P(O)(OR⁶)(OR⁷), —P(O)₂(OR⁶)M, or —P(O)₃MY, wherein R⁶ and R⁷ are each independently selected from hydrogen, methyl, ethyl, propyl, butyl, pentyl, or hexyl, M and Y are each independently selected from sodium ions, potassium ions, or MY is a divalent cation selected from calcium ions, magnesium ions.

In one embodiment, R¹ is selected from H, methyl, ethyl, or propyl; R² is selected from H, —OR³, substituted or unsubstituted furanyl, substituted or unsubstituted thienyl, substituted or unsubstituted pyrrolyl, substituted or unsubstituted imidazolyl, substituted or unsubstituted pyrazolyl, substituted or unsubstituted thiazolyl, substituted or unsubstituted oxazolyl, substituted or unsubstituted pyridyl, substituted or unsubstituted pyrimidyl, substituted or unsubstituted pyridazinyl, substituted or unsubstituted pyrazinyl, substituted or unsubstituted purinyl, substituted or unsubstituted quinolyl, substituted or unsubstituted isoquinolyl, substituted or unsubstituted indolyl,

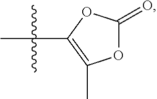 [SMILES: *c1oc(=O)oc1C]

wherein the aforementioned substituted substituent is selected from methyl, ethyl, or propyl;

R³ is selected from —C(O)(CH)(R⁴)(R⁵), —P(O)(OR⁶)(OR⁷), —P(O)₂(OR⁶)M, or —P(O)₃MY;

R⁴ is selected from hydrogen, amino, methyl, ethyl, or propyl;

R⁵ is selected from hydrogen, methyl, ethyl, propyl, butyl, pentyl, hexyl, heptyl, octyl, nonyl, decyl, tetrahydrofuranyl, tetrahydropyrrolyl, furanyl, thienyl, pyrrolyl, imidazolyl, pyrazolyl, thiazolyl, oxazolyl, pyridyl, pyrimidyl, pyridazinyl, pyrazinyl, purinyl, quinolyl, isoquinolyl, indolyl, phenyl, benzyl, —(CH₂)nSCH₃,—(CH₂)mNHCH₃, or —(CH₂)mN(CH₃)_{2;}

R⁶ and IC are each independently selected from hydrogen, methyl, ethyl, or propyl; M and Y are each independently selected from sodium ions, potassium ions, or MY is a divalent cation selected from calcium ions, magnesium ions; and

m and n are each independently selected from 1, 2, 3, 4, 5 or 6.

In a preferred embodiment,wherein,

R¹ is selected from H or methyl;

R² is selected from H, —OR³,

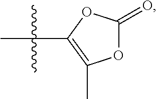 [SMILES: *c1oc(=O)oc1C]

wherein the substituent is selected from methyl, ethyl, or propyl;

R³ is selected from —C(O)(CH)(R⁴)(R⁵), —P(O)(OR⁶)(OR⁷), —P(O)₂(OR⁶)M, or —P(O)₃MY;

R⁴ is selected from hydrogen or amino;

R⁵ is selected from hydrogen, methyl, ethyl, propyl, butyl, pentyl, hexyl, heptyl, octyl, nonyl, decyl, tetrahydrofuranyl, tetrahydropyrrolyl, furanyl, thienyl, pyrrolyl, imidazolyl, pyrazolyl, thiazolyl, oxazolyl, pyridyl, pyrimidyl, pyridazinyl, pyrazinyl, purinyl, quinolyl, isoquinolyl, indolyl, phenyl, benzyl, —(CH₂)nSCH₃, —(CH₂)mNHCH₃, or —(CH₂)mN(CH₃)₂;

R⁶ and R⁷ are hydrogen; M and Y are each independently selected from sodium ions, potassium ions, or MY is a divalent cation selected from calcium ions, magnesium ions; and

m and n are each independently selected from 1, 2, 3, 4, 5 or 6.

According to the present invention, the compounds of general formula (I) thereof preferably includes:

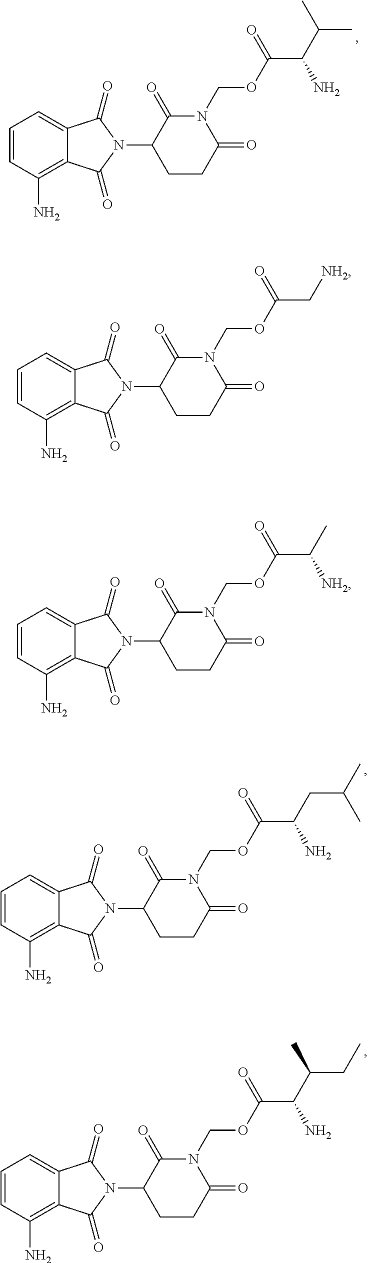

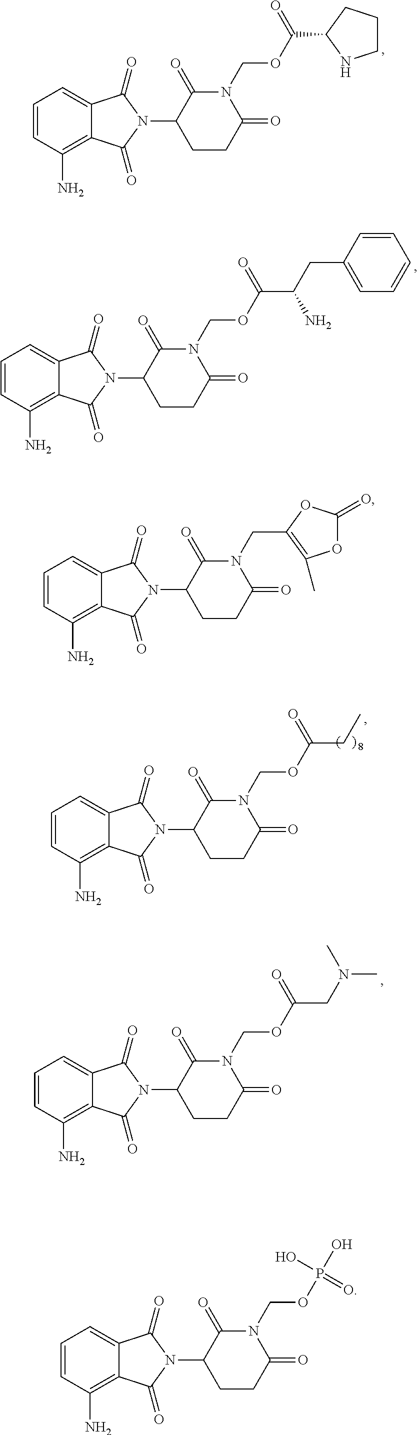

The pharmaceutically acceptable salt as described in the present invention includes, but are not limited to, hydrochloride or tromethamine salt.

In another aspect, the present invention provides a pharmaceutical composition comprising a therapeutically effective amount of the compound (including a stereoisomer thereof) as described above, or a pharmaceutically acceptable salt thereof, and a pharmaceutically acceptable carrier and/or excipient.

In a further aspect, the present invention provides the use of the compound as described above (including a stereoisomer thereof) or a pharmaceutically acceptable salt thereof in preparing a medicament for the treatment of cancer. The cancer includes, but are not limited to, multiple myeloma, prostate cancer.

In the present invention, unless otherwise specified, the meanings of the terms used are as follows.

The compound represented by formula (I) according to the present invention includes a stereoisomer thereof.

The carbon, hydrogen, oxygen, sulfur, nitrogen or halogen referred to in the groups and compounds of the present invention include their isotopes, and the carbon, hydrogen, oxygen, sulfur, nitrogen or halogen referred to in the groups and compounds of the present invention is optionally further replaced by one or more of their corresponding isotopes, wherein the carbon isotopes include ¹³C and ¹⁴C, the hydrogen isotopes include protium (H), deuterium (D, also known as heavy hydrogen), and tritium (T, also known as super heavy hydrogen), the oxygen isotopes include ¹⁶O, ¹⁷O and ¹⁸O,the sulfur isotopes include ³²S, ³³S, ³⁴S and ³⁶S, the nitrogen isotopes include ¹⁴N and ¹⁵N, the fluorine isotopes include ¹⁹F, the chlorine isotopes include ³⁵Cl and ³⁷Cl,and the bromine isotopes include ⁷⁹Br and ⁸¹Br.

“Alkyl” indicates a straight-chain or branched-chain saturated aliphatic hydrocarbonyl group, with the main chain comprising from 1 to 20 carbon atoms, preferably from 1 to 12 carbon atoms, further preferably from 1 to 8 carbon atoms, more preferably from 1 to 6 carbon atoms, still more preferably straight-chain and branched-chain groups with from 1 to 4 carbon atoms, and most preferably from 1 to 2 carbon atoms; examples of alkyl groups include, but are not limited to, methyl, ethyl, n-propyl, isopropyl, n-butyl, isobutyl, sec-butyl, tert-butyl, n-pentyl, 2-pentyl, 3-pentyl, 2-methyl-2-butyl, 3-methyl-2-butyl, 3-methyl-1-butyl, 2-methyl-1-butyl, n-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, n-heptyl, 2-methylhexyl, 3-methylhexyl, 4-methylhexyl, 5-methylhexyl, 2,2-dimethylpentyl, 2,3-dimethylpentyl, 2,4-dimethylpentyl, 3,3-dimethylpentyl, 2-ethylpentyl, 3-ethylpentyl, n-octyl, 2,2-dimethylhexyl, 2,3-dimethylhexyl, 2,4-dimethylhexyl, 2,5-dimethylhexyl, 3,3-dimethylhexyl, 4,4-dimethylhexyl, 2-ethylhexyl, 3-ethylhexyl, 4-ethylhexyl, 2-methyl-2-ethylpentyl, 2-methyl-3-ethylpentyl, n-nonyl, 2-methyl-2-ethylhexyl, and n-decyl.

“Alkoxy” indicates an -O-alkyl group in which the alkyl is as defined above. Alkoxy may be substituted or unsubstituted, and examples of alkoxy include, but are not limited to, methoxy, ethoxy, n-propoxy, isopropoxy, n-butoxy, isobutoxy, tert-butoxy, sec-butoxy, n-pentyloxy, and n-hexyloxy.

“Alkenyl” indicates an alkyl group as defined above containing at least one carbon-carbon double bond, preferably containing from 2 to 20 carbon atoms, further preferably from 2 to 12 carbon atoms, and more preferably containing from 2 to 8 carbon atoms in the main chain, while the alkenyl group may be substituted or unsubstituted; non-limiting examples include vinyl, allyl, 1-propenyl, 2-propenyl, 1-butenyl, 2-butenyl, 3-butenyl, 1-pentenyl, 2-pentenyl, 3-pentenyl, 4-pentenyl, 1-methyl-1-butenyl, 2-methyl-1-butenyl, 2-methyl-3-butenyl, 1-hexenyl, 2-hexenyl, 3-hexenyl, 4-hexenyl, 5-hexenyl, 1-methyl-1-pentenyl, 2-methyl-1-pentenyl, 1-heptenyl, 2-heptenyl, 3-heptenyl, 4-heptenyl, 1-octenyl, 3-octenyl, 1-nonenyl, 3-nonenyl, 1-decenyl, 4-decenyl, 1,3-butadiene, 1,3-pentadiene, 1,4-pentadiene, 1,4-hexadiene, 3-undecylenyl, 4-dodecenyl, and 4,8,12-tetradecatrienyl.

“Alkynyl” indicates an alkyl group as defined above containing at least one carbon-carbon triple bond, preferably containing from 2 to 20 carbon atoms, further preferably from 2 to 8 carbon atoms, and more preferably an alkynyl group containing from 2 to 4 carbon atoms in the main chain, while the alkenyl group may be substituted or unsubstituted; and non-limiting examples include ethynyl, 1-propinyl, 2-propinyl, butynyl, 2-butynyl, 3-butynyl, 1-methyl-2-propinyl, 4-pentynyl, 3-pentynyl, 1-methyl-2-butynyl, 2-hexynyl, 3-hexynyl, 2-heptynyl, 3-heptynyl, 4-heptynyl, 3-octynyl, 3-nonynyl, 4-decynyl, 3-undecynyl, and 4-dodecynyl.

“Cycloalkyl” indicates a ring of carbon monocyclic, fused, spiro or bridged rings, including, but not limited to, cyclopropane, cyclobutane, cyclopentane, spiro[3.4]octane, bicyclo[3.1.1]hexane, etc.

“Heterocycle” or “heterocyclyl” indicates a substituted or unsubstituted saturated or unsaturated non-aromatic ring system containing at least 1 to 5 atoms or groups selected from N, O, S, S(═O) or S(═O)₂, wherein the non-aromatic ring system contains from 3 to 20 ring atoms, preferably from 3 to 10 ring atoms, and more preferably from 3 to 8 ring atoms; the selectively substituted N and S in the heterocyclyl ring can be oxidized to various oxidation states; and non-limiting examples include oxacyclopropanyl, oxacyclobutyl, oxacyclopentyl, oxacyclohexyl, oxacyclohexyl, oxacyclooctyl, azacyclopropanyl, azacyclobutyl, azacyclopentyl, azacyclohexyl, azacyclopropenyl, 1,3-dioxacyclopentyl, 1,4-dioxacyclopentyl, 1,3-dioxacyclopentyl, 1,3-dioxacyclohexyl, 1,3-dithiocyclohexyl, azacycloheptenyl, morpholinyl, piperazinyl, pyridyl, furanyl, thienyl, pyrrolyl, pyranyl, N-alkylpyrrolyl, pyrimidyl, pyrazinyl, pyridazinyl, imidazolyl, piperidinyl, thiomorpholinyl, dihydropyran, thiadiazolyl, oxazolyl, oxadiazolyl, pyrazolyl, 1,4-dioxacyclohexadienyl, 2H-1,2-oxazinyl or 2,5-dihydrothienyl, etc.

“Heteroaryl” indicates a substituted or unsubstituted 5- to 14-membered aromatic ring (preferably a 5- to 10-membered heteroaromatic ring, and more preferably 5- to 6-membered), and contains 1 to 5 atoms or groups selected from N, O or S(═O)n; non-limiting examples of heteroaryl groups include, but are not limited to, pyridyl, furanyl, thienyl, pyridyl, pyranyl, N-alkylpyrrolyl, pyrimidyl, pyrazinyl, pyridazinyl, imidazolyl, piperidinyl, morpholine, thiomorpholine, 1,3-dithiane, benzimidazole, piperidinyl, benzimidazole, benzopyridine, and pyrrolopyridine; and the heteroaryl ring can be fused to an aryl, heterocyclyl or cycloalkyl ring, wherein the ring to which the parent structure is attached is a heteroaryl ring.

“Substituted” refers to the case where one or more hydrogen atoms in a group are substituted by another group, and if the group is substituted by a hydrogen atom, the group formed is the same as the group substituted by the hydrogen atom.

“Substituted or unsubstituted” refers to the case where a group may or may not be substituted, and if it is not indicated in the present invention that a group may be substituted, this means that the group is unsubstituted.

“Each independently selected from” means that the various substituents may be the same or different, and even different substituents represented by the same substituent symbol in the same embodiment may be the same or different.

“Pharmaceutically acceptable salt” refers to those which retain the biological effectiveness and properties of the parent compound, said salts are obtained through the reaction of the free acid of the parent compound with an inorganic base or organic base, or obtained through the reaction of the free base of the parent compound with an inorganic acid or organic acid.

### BRIEF DESCRIPTION OF THE DRAWINGS

FIG. 1 shows the relationship between drug concentrations in plasma and the time in rats after the administration of each group of compounds.

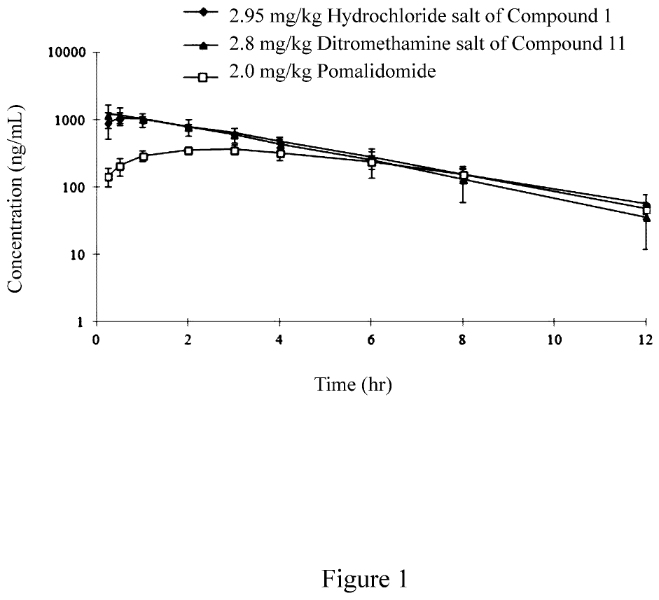

### DETAILED DESCRIPTION OF THE INVENTION

The present invention will be further clarified by the following examples, and may allow a person skilled in the art a way to more fully understand, without limiting the present invention in any way.

### EXAMPLE 1

Preparation of Hydrochloride Salt of Compound 1

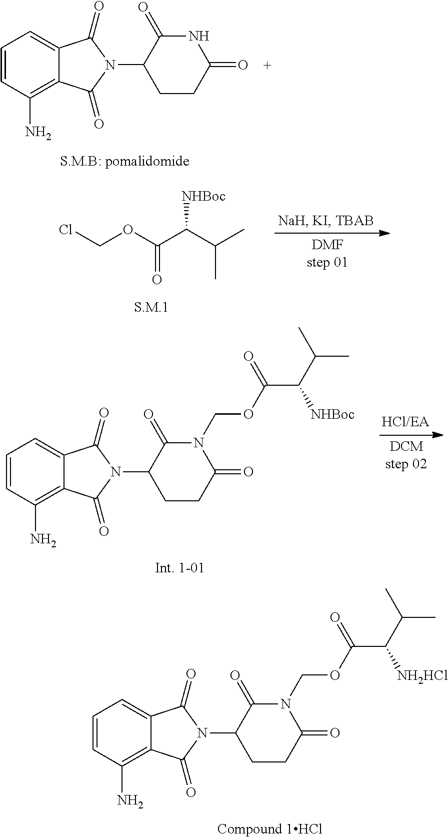

Step 1: Under nitrogen protection, S.M.B (20 g, 73.2 mmol, 1.00 eq) and DMF (400 mL) were added to a 1000 mL three-neck reaction flask, and stirred. Sodium hydride (3.5 g, 87.5 mmol, 1.2 eq) was then added slowly; after stirring for 30 min, potassium iodide (12 g, 72.2 mmol, 0.99 eq) and TBAB (tetrabutylammonium bromide) (3.52 g, 10.9 mmol, 0.15 eq) were added; and after stirring for 15 min, S.M.1 (23.8 g, 72.8 mmol, 0.99 eq) was added and stirred for 12 h. The reaction was stopped, and the reaction system was poured into 2 L of water, stirred for 1 h, and solids were precipitated and filtered to obtain a yellow solid filter cake, the filter cake was washed with 500 mL of DCM (dichloromethane) for liquid separation, and the organic phase was concentrated under reduced pressure and purified by column chromatography (PE:EA=1.5:1) to give 13.20 g of a yellow solid product with a yield of 35.8%.

Step 2: Under nitrogen protection, Int 1-01 (500 mg, 1 mmol, 1.00 eq) and DCM (5 mL) were added to a 100 mL 3-neck reaction flask, and after stirring at room temperature for 10 min, HCl/EA (10 mL, 10 mmol, 10.0 eq) was added once and stirring was continued for another 40 min. The reaction was stopped and subjected to filtration,the filter cake was dried to obtain 380 mg of a yellow solid product with a yield of 86.9%.

HPLC: 97.05%

LCMS:403.2(M+H-HCl)

¹H NMR (400 MHz, DMSO) δ 8.56-8.51 (d, 3H), 7.51-7.47 (dd, 1H), 7.06-7.04 (d, 1H), 7.01-7.02 (d, 1H), 6.56 (bs, 2H), 5.91-5.85 (dd, 1H), 5.74-5.69 (t, 1H), 5.28-5.23 (m, 1H), 3.92 (s, 1H), 2.25 (m, 1H), 2.88-2.84 (d, 1H), 2.61-2.51 (m, 1H), 2.16-2.10 (m, 2H), 0.96-0.92 (m, 6H).

### EXAMPLE 2

Preparation of Hydrochloride Salt of Compound 2

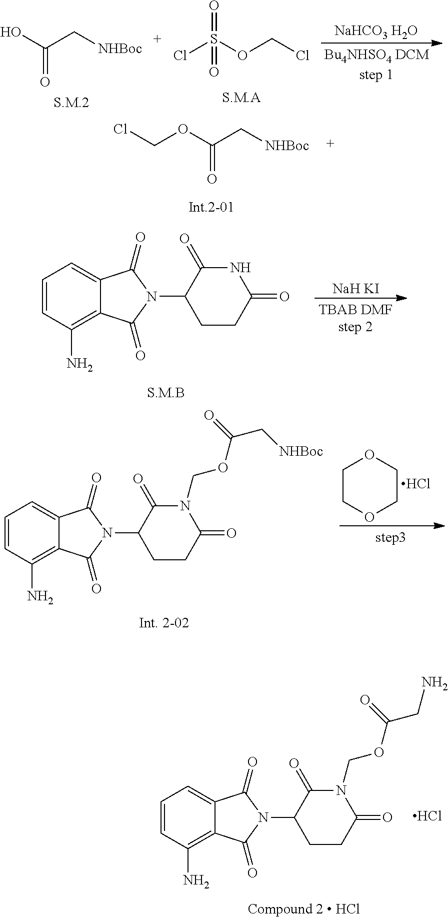

Step 1: S.M.2 (50 g, 285.4 mmol, 1.00 eq), H₂O (1000 mL), and sodium bicarbonate (95.9 g, 1141.6 mmol, 4.00 eq) were added to a 2000 mL 3-neck reaction flask; after stirring for 30 min, DCM and Bu₄NHSO₄ (9.7 g, 28.5 mmol, 0.1 eq) were added; and after stirring for another 20 min, S.M.A (56.5 g, 342.5 mmol, 1.2 eq) was added and the reaction was stirred for 15 h. The reaction was stopped and subjected to liquid separation, and the organic phase was concentrated under reduced pressure to give 60 g of a colorless oil with a yield of 93.8%.

Step 2: Undernitrogen protection, S.M.B (5.0 g, 18.3 mmol, 1.00 eq) and DMF (50 mL) were added to a 100 mL single-mouth reaction flask, and were stirred for 10 min in an ice water bath; NaH (0.9 g, 22.0 mmol, 1.2 eq) was then added once; after stirring for 20 min, TBAB (0.6 g, 1.8 mmol, 0.1 eq) and KI (3.0 g, 18.3 mmol, 1.00 eq) were added; and after reacting for 10 min in an ice water bath, Int. 2-01 (6.2 g, 27.5 mmol, 1.50 eq) was added and same was stirred at room temperature overnight. The reaction was stopped and subjected to liquid separation, and the organic phase was concentrated under reduced pressure and purified by column chromatography (DCM:EA=5:1) to give 1.5 g of product.

Step 3: Under nitrogen protection, Int 2-02 (0.1 g, 0.2 mmol, 1.00 eq) and hydrochloric acid/dioxane (20 mL) were added to a 25 mL three-mouth reaction flask, and stirred at room temperature overnight. The reaction was stopped and drying by filtration was performed to give 73 mg of a yellow solid product with a yield of 92.4%. HPLC: 96.24%

LCMS:361.1(M+H⁺)

¹H NMR (400 MHz, DMSO) δ=8.50 (s, 2H), 7.49 (t, 1H), 7.04 (dd, 2H), 5.79 (q, J=9.7, 2H), 5.24 (dd, 4.5, 1H), 3.82 (d, 2H), 3.57 (s, 1H), 3.14-3.01 (m, 1H), 2.85 (d, 1H), 2.60 (d, 1H).

### EXAMPLE 3

Preparation of Hydrochloride Salt of Compound 3

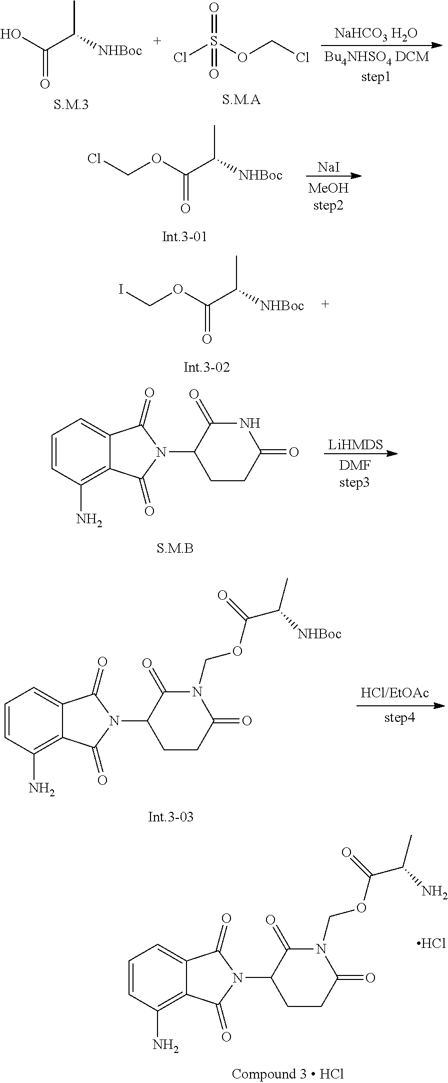

Step 1: S.M.1 (6 g, 31.7 mmol, 1.00 eq), H₂O (130 mL), and sodium bicarbonate (10.6 g, 126.8 mmol, 4.00 eq) were added to a 500 mL 3-neck reaction flask and stirred for 30 min; then DCM and Bu₄NHSO₄ (1.08 g, 3.2 mmol, 0.1 eq) were added and stirred for 20 min; and S.M.A (6.3 g, 38.0 mmol, 1.2 eq) was added and the reaction was stirred for 15 h. The reaction was stopped and subjected to liquid separation, and the organic phase was concentrated under reduced pressure to give 7.2 g of a colorless oil with a yield of 95.5%.

Step 2: Int 2-02 (5.0 g, 18.3 mmol, 1.00 eq), NaI (18 g, 121 mmol) and MeCN (75 mL) were added to a 50 mL single-mouth reaction flask, and stirred at room temperature overnight. The reaction was stopped and subjected to liquid separation, and the organic phase was concentrated under reduced pressure to give 9.2 g of a colorless oil with a yield of 92.2%.

Step 3: Under nitrogen protection, S.M.B (3 g, 10.98 mmol, 1.00 eq) and DMF (65 mL) were added to a 250 mL three-mouth reaction flask; the reaction system was cooled down to below −20° C., and LiHMDS (11 mL, 11 mmol, 1.00 eq) was added dropwise; and after stirring for 10 min, a solution of Int. 3-02 (4.34 g, 13.17 mmol, 1.20 eq) in DMF was added and stirring was continued. The reaction was stopped, the system was poured into a 1% NH₄Cl (300 mL) ice water solution, and the solid was precipitated with stirring, the solid was filtered, and the filter cake was dissolved in EtOAc, concentrated under reduced pressure and purified by column chromatography (PE/EtOAc=10:1-2:1) to give 1.6 g of product with a yield of 30%.

Step 4: Under nitrogen protection, Int 3-03 (0.5 g, 1.05 mmol, 1.00 eq) and hydrochloric acid/dioxane (20 mL) were added to a 25 mL three-mouth reaction flask, and stirred at room temperature overnight. The reaction was stopped and subjected to filtration, the filter cake was dried to obtain 235 mg of a yellow solid product with a yield of 54.2%. HPLC: 96.4%

LCMS:375.1(M+H⁺)

¹H NMR (400 MHz, DMSO-d6) δ 8.56 (s, 1H), 7.48 (t, J=7.8 Hz, 1H), 7.05 (d, J=8.4 Hz, 1H), 7.01 (d, J=7.1 Hz, 1H), 5.86 (t, J=9.1 Hz, 1H), 5.74 (d, J=9.5 Hz, 1H), 5.24 (dd, J=13.0, 5.3 Hz, 1H), 4.11-4.02 (m, 3H), 3.11-3.03 (m, 3H), 2.87-2.74 (m, 1H), 2.68-2.57 (m, 1H), 2.14-2.08 (m, 1H), 1.38 (d, J=7.0 Hz, 3H).

### EXAMPLE 4

Preparation of Hydrochloride Salt of Compound 4

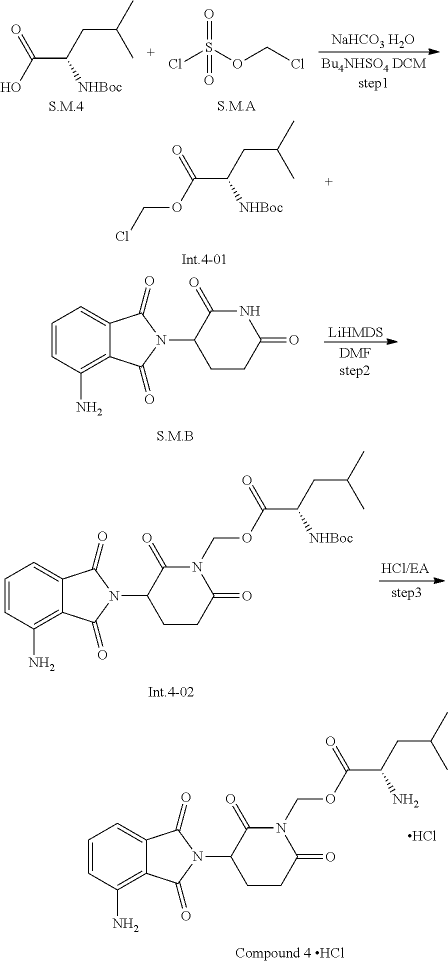

Step 1: S.M.4 (6.3 g, 25.27 mmol, 1.00 eq), H₂O (110 mL), and sodium bicarbonate (8.48 g, 101.08 mmol, 4.00 eq) were added to a 500 mL 3-neck reaction flask and stirred for 10 min; then DCM and Bu₄NHSO₄ (0.86 g, 2.52 mmol, 0.1 eq) were added and stirred for 10 min; S.M.A (5 g, 30.32 mmol, 1.2 eq) was added dropwise under an ice bath condition and the reaction was stirred at room temperature for 15 h. The reaction was stopped and subjected to liquid separation, and the organic phase was concentrated under reduced pressure to give 7.4 g of colorless oil with a yield of 98.4%.

Step 2: Under nitrogen protection, S.M.B (2.7 g, 9.88 mmol, 1.00 eq) and DMF (70 mL) were added to a 250 mL three-mouth reaction flask; the reaction system was cooled down to below −20° C., and LiHMDS (9.88 mL, 9.88 mmol, 1.00 eq) was added dropwise; and after stirring for 10 min, a solution of Int. 4-01 (3.8 g, 12.84 mmol, 1.3 eq) in DMF was then added, and stirring was continued at a low temperature. The reaction was stopped, the reaction system was poured into a 1% NH₄Cl (315 mL) ice water solution, and the solid was precipitated with stirring, the solid was filtered, and the filter cake was dissolved in EA, concentrated under reduced pressure and purified by column chromatography (PE:EA=2:1) to give 800 mg of product with a yield of 15%.

Step 3: Under nitrogen protection, Int 4-02 (0.2 g, 0.37 mmol, 1.00 eq) and 2N hydrochloric acid/EA (4 mL/4 mL) were added to a 25 mL three-mouth reaction flask, and stirred at room temperature overnight. The reaction was stopped and subjected to filtration, the filter cake was dried to obtain 80 mg of a yellow solid product with a yield of 50%.

HPLC: 98.24%.

LCMS:417.4(M+H⁺)

¹H NMR (400 MHz, DMSO) δ 8.43 (s, 2H), 7.50 (t, J=7.7 Hz, 1H), 7.09-6.95 (m, 2H), 6.55 (s, 2H), 5.87 (d, J=9.6 Hz, 1H), 5.74 (d, J=9.8 Hz, 1H), 5.24 (d, J=12.9 Hz, 1H), 4.00 (s, 1H), 3.09 (t, J=15.2 Hz, 1H), 2.86 (d, J=18.2 Hz, 1H), 1.76-1.64 (m, 1H), 1.57 (dd, J=14.0, 6.9 Hz, 2H), 1.24 (s, 2H), 0.89 (d, J=6.2 Hz, 6H).

### EXAMPLE 5

Preparation of Hydrochloride Salt of Compound 5

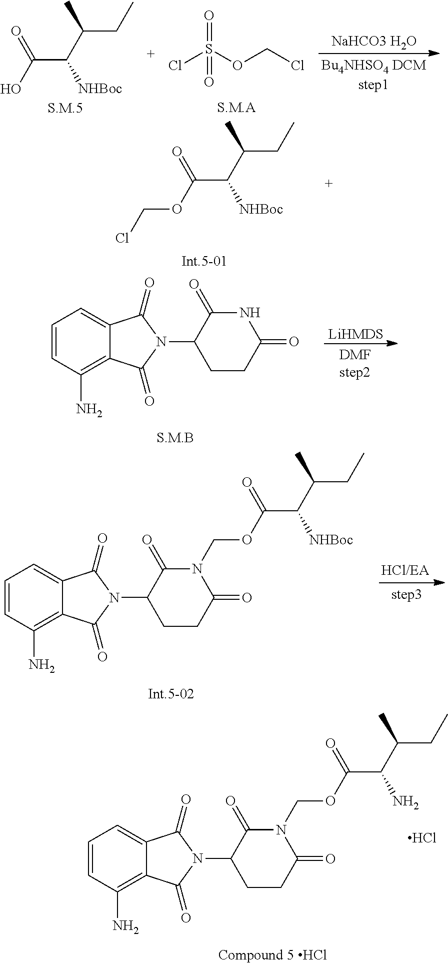

Step 1: The preparation method was the same as that of Step 1 of Example 4, except that S.M.4 was changed to S.M.5. 7.6 g of a colorless oil was obtained with a yield of 99.2%.

Step 2: The preparation method was the same as that of Step 2 of Example 4, except that Int. 4-01 was changed to Int. 5-01. 800 mg of product was obtained with a yield of 15%.

Step 3: The preparation method was the same as that of Step 3 of Example 4, except that Int. 4-02 was changed to Int. 5-02. 83.6 mg of a yellow solid product was obtained with a yield of 52%.

HPLC: 99.5%

LCMS:417.4(M+H⁺)

¹H NMR (400 MHz, DMSO) δ 8.47 (s, 3H), 7.49 (s, 1H), 7.05 (d, J=8.5 Hz, 2H), 6.52 (s, 2H), 5.87 (d, J=9.8 Hz, 1H), 5.71 (dd, J=9.7, 4.3 Hz, 1H), 5.29-5.16 (m, 1H), 3.97 (s, 1H), 3.08 (ddd, J=18.7, 12.7, 6.1 Hz, 1H), 2.86 (d, J=17.5 Hz, 1H), 2.63 (dd, J=26.7, 13.6 Hz, 1H), 2.20-2.05 (m, 1H), 1.41 (dt, J=13.3, 6.6 Hz, 1H), 1.32-1.07 (m, 2H), 0.93-0.70 (m, 6H).

LCMS:417.4(M+H⁺)

### EXAMPLE 6

Preparation of Hydrochloride Salt of Compound 6

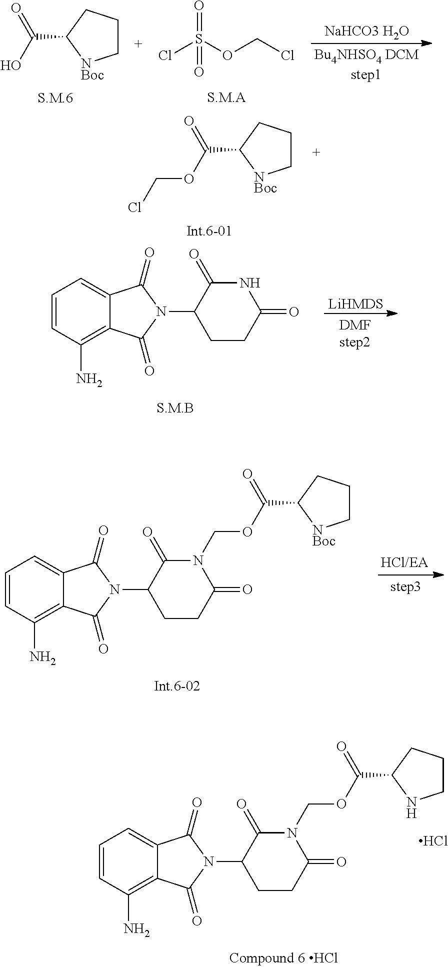

Step 1: The preparation method was the same as that of Step 1 of Example 4, except that S.M.4 was changed to S.M.6. 6.3 g of a colorless oil was obtained with a yield of 94.2%.

Step 2: The preparation method was the same as that of Step 2 of Example 4, except that Int. 4-01 was changed to Int. 6-01. 100 mg of product was obtained with a yield of 2%.

Step 3: The preparation method was the same as that of Step 3 of Example 4, except that Int. 4-02 was changed to Int. 6-02. 45 mg of a yellow solid product was obtained with a yield of 56.2%.

HPLC: 96.53%

LCMS:401.4(M+H⁺)

¹H NMR (400 MHz, DMSO) δ 10.00 (s, 1H), 9.00 (s, 1H), 7.48 (s, 1H), 7.03 (dd, J=11.8, 7.8 Hz, 2H), 6.53 (s, 2H), 5.86 (s, 2H), 5.24 (dd, J=13.0, 5.2 Hz, 1H), 4.40 (s, 1H), 3.16 (d, J=32.7 Hz, 2H), 3.13-2.98 (m, 1H), 2.91-2.78 (m, 1H), 2.22 (dd, J=14.8, 7.1 Hz, 1H), 2.16-2.04 (m, 1H), 1.91 (s, 4H), 0.85 (s, 0H).

### EXAMPLE 7

Preparation of Hydrochloride Salt of Compound 7

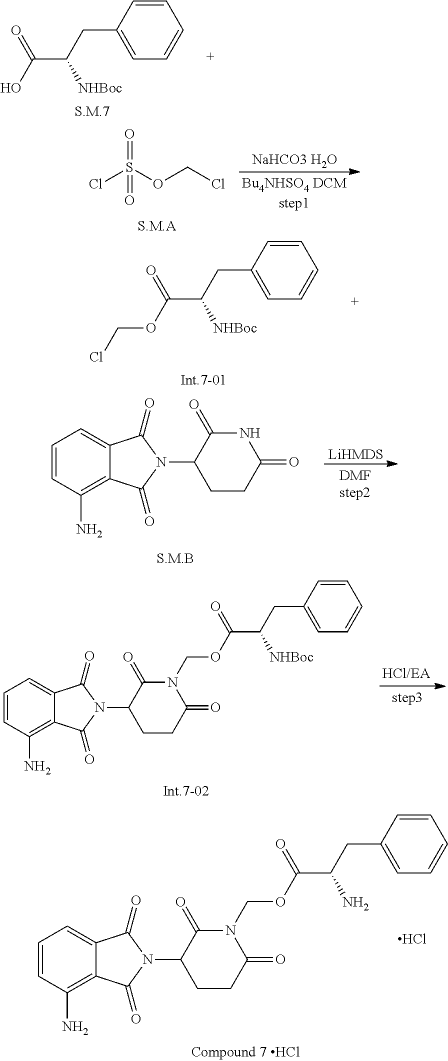

Step 1: The preparation method was the same as that of Step 1 of Example 4, except that S.M.4 was changed to S.M.7. 7 g of a colorless oil was obtained with a yield of 88.6%.

Step 2: The preparation method was the same as that of Step 2 of Example 4, except that Int. 4-01 was changed to Int. 7-01. 2.1 g of product was obtained with a yield of 40.2%.

Step 3: The preparation method was the same as that of Step 3 of Example 4, except that Int. 4-02 was changed to Int. 7-02. 45 mg of a yellow solid product was obtained with a yield of 56.2%.

HPLC: 96.41%

LCMS:451.4(M+H⁺)

¹H NMR (400 MHz, DMSO) δ 8.64 (s, 3H), 7.48 (dd, J=10.3, 4.3 Hz, 1H), 7.38-7.15 (m, 5H), 7.14-6.92 (m, 2H), 5.94-5.78 (m, 1H), 5.66 (dd, J=9.6, 3.1 Hz, 1H), 5.20 (d, J=13.0 Hz, 1H), 4.33 (s, 1H), 3.09 (dt, J=21.8, 11.7 Hz, 3H), 2.86 (d, J=16.2 Hz, 1H), 2.77-2.51 (m, 1H), 2.12 (d, J=6.2 Hz, 1H).

### EXAMPLE 8

Preparation of Compound 8

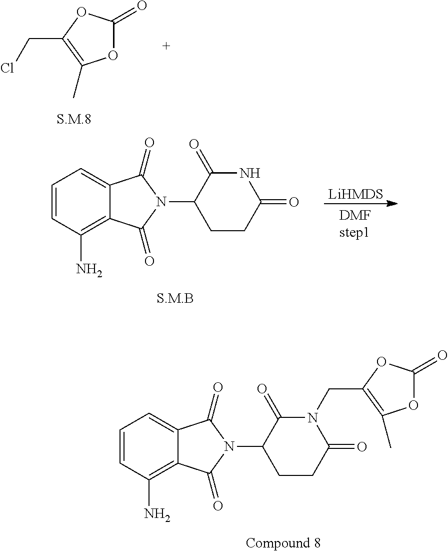

Under nitrogen protection, S.M.B (2.5 g, 9.15 mmol, 1.00 eq) and DMF (65 mL) were added to a 250 mL three-mouth reaction flask, the reaction system was cooled down to below −20° C., LiHMDS (9.15 mL, 9.15 mmol, 1.00 eq) was added dropwise, and stirring was continued for 10 min at a low temperature, and a solution of S.M.8 (2.72 g, 11.89 mmol, 2.00 eq) in DMF was then added and the reaction was carried out in an ice bath. The reaction was stopped, the reaction system was poured into a 1% NH₄Cl (300 mL) ice water solution, and the solid was precipitated with stirring, the solid was filtered, and the filter cake was dissolved in EtOAc, concentrated under reduced pressure and purified by column chromatography (PE:EtOAc=2:1) to give 290 mg of product.

HPLC: 82.4%

LCMS:386.1(M+H⁺)

¹H NMR (400 MHz, DMSO) δ 8.64 (s, 3H), 7.48 (dd, J=10.3, 4.3 Hz, 1H), 7.38-7.15 (m, 5H), 7.14-6.92 (m, 2H), 5.94-5.78 (m, 1H), 5.66 (dd, J=9.6, 3.1 Hz, 1H), 5.20 (d, J=13.0 Hz, 1H), 4.33 (s, 1H), 3.09 (dt, J=21.8, 11.7 Hz, 3H), 2.86 (d, J=16.2 Hz, 1H), 2.77-2.51 (m, 1H), 2.12 (d, J=6.2 Hz, 1H).

### EXAMPLE 9

Preparation of Compound 9

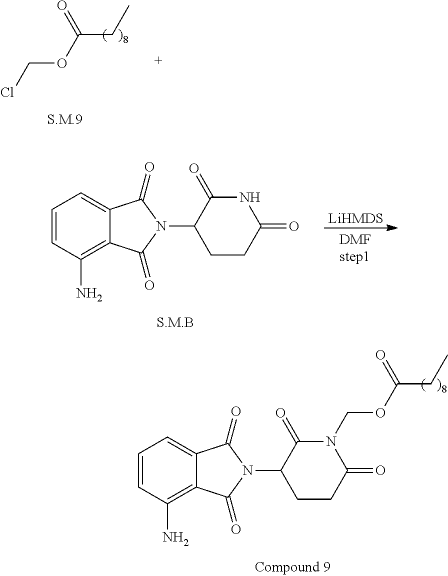

Step 1: Under nitrogen protection, S.M.B (1 g, 3.66 mmol, 1.00 eq) and DMF (25 mL) were added to a 100 mL three-mouth reaction flask, the reaction system was cooled down to below −20° C., LiHMDS (3.66 mL, 3.66 mmol, 1.00 eq) was added dropwise, and stirred for 10 min, and a solution of S.M.9 (1.21 g, 5.49 mmol, 1.20 eq) in DMF was then added and the reaction was carried out in an ice water bath. The reaction was stopped, the reaction system was poured into a 1% NH₄Cl (120 mL) ice water solution, and the solid was precipitated with stirring, the solid was filtered, and the filter cake was dissolved in EtOAc, concentrated under reduced pressure and purified by column chromatography (PE:EA=2:1) to give 310 mg of product with a yield of 18.3%.

HPLC: 98.2%

LCMS:480.2(M+Na⁺)

¹H NMR (400 MHz, DMSO) δ 7.48 (t, J=7.7 Hz, 1H), 7.02 (t, J=8.1 Hz, 2H), 6.53 (s, 2H), 5.64 (d, J=2.3 Hz, 2H), 5.23 (dd, J=13.0, 5.3 Hz, 1H), 3.05 (td, J=13.9, 13.4, 6.9 Hz, 1H), 2.82 (dt, J=17.8, 3.2 Hz, 1H), 2.72-2.51 (m, 2H), 2.27 (t, J=7.3 Hz, 2H), 2.15-2.02 (m, 1H), 1.49 (t, J=7.1 Hz, 2H), 1.22 (s, 1H), 0.84 (t, J=6.6 Hz, 3H).

### EXAMPLE 10

Preparation of Hydrochloride Salt of Compound 10

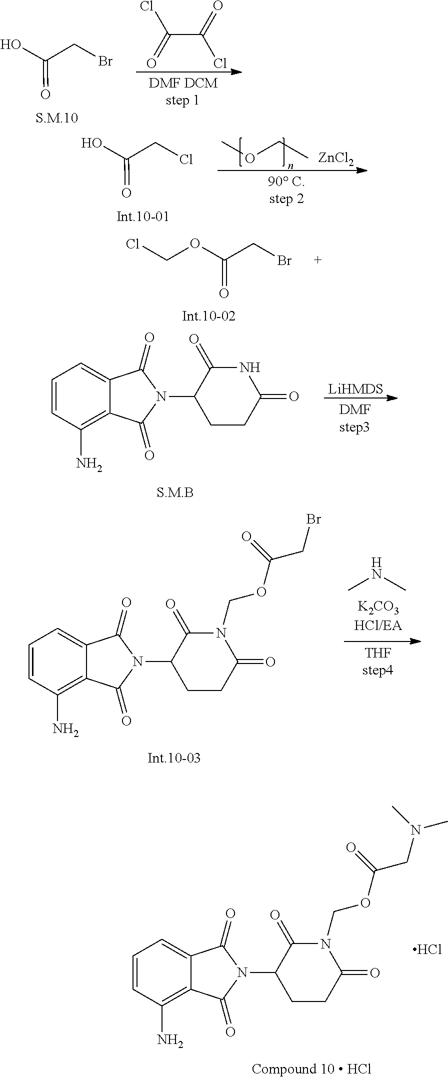

Step 1: Under nitrogen protection, S.M.10 (15 g, 107.9 mmol, 1.00 eq), DCM (150 mL) and DMF (4 drops) were added to a 500 mL 3-neck reaction flask, and oxalyl chloride (15.07 g, 118.74 mmol, 1.1 eq) was further added dropwise under an ice bath condition and stirred at room temperature for 2 h. The reaction was stopped and the reaction solution was concentrated under reduced pressure to give 14.56 g of oils with a yield of 86.1%.

Step 2: Int. 10-01 (14.56 g, 92.06 mmol, 1.00 eq), paraformaldehyde (2.78 g, 92.06 mmol, 1.00 eq) and zinc chloride (2.52 g, 18.41 mmol, 0.2 eq) were added to a 100 mL single-mouth reaction flask, and the reaction system was warmed to 90° C. and stirred. The reaction was stopped and the reaction solution was concentrated under reduced pressure and subjected to column chromatography to give 4.3 g of product with a yield of 24.7%.

Step 3: Under nitrogen protection, S.M.B (4.48 g, 16.4 mmol, 1.00 eq) and DMF (65 mL) were added to a 250 mL three-mouth reaction flask, the reaction system was cooled down to below −20° C., LiHMDS (16.4 mL, 16.4 mmol, 1.00 eq) was added dropwise, and stirred for 10 min, and a solution of Int. 10-02 (4.0 g, 21.3 mmol, 1.3 eq) in DMF was then added and stirred in an ice bath. The reaction system was poured into a 1% NH₄Cl (360 mL) ice water solution, and the solid was precipitated with stirring, the solid was filtered, and the filter cake was dissolved in EA, concentrated under reduced pressure and purified by column chromatography (DCM:EA=50:1-30:1) to give 3.3 g of product with a yield of 47.5%.

Step 4: Int. 10-03 (3.33 g, 7.85 mmol, 1.00 eq), THF 66 mL, dimethylamine (1.41 g, 31.4 mmol, 4.00 eq) and KI (33 mg) were added to a 250 mL three-mouth reaction flask, and stirred at room temperature. The reaction was stopped, and the system was directly spin-dried with a reduced pressure pump and dissolved in a small amount of EA, and then salted out with 3N HCl/EA, and was then filtered to give 500 mg of product with a yield of 15%.

HPLC: 98.6%

LCMS:451.4(M+H⁺)

¹H NMR (400 MHz, DMSO) δ 10.78 (s, 0H), 7.48 (dd, J=8.4, 7.1 Hz, 1H), 7.04 (dd, J=21.7, 7.6 Hz, 2H), 6.59 (s, 2H), 5.81 (q, J=9.7 Hz, 1H), 5.26 (dd, J=13.0, 5.4 Hz, 1H), 3.61-3.18 (m, 1H), 2.83 (s, 3H), 2.51 (dt, J=3.8, 1.9 Hz, 1H), 2.22-1.98 (m, 1H).

### EXAMPLE 11

Preparation of Ditromethamine salt of Compound 11

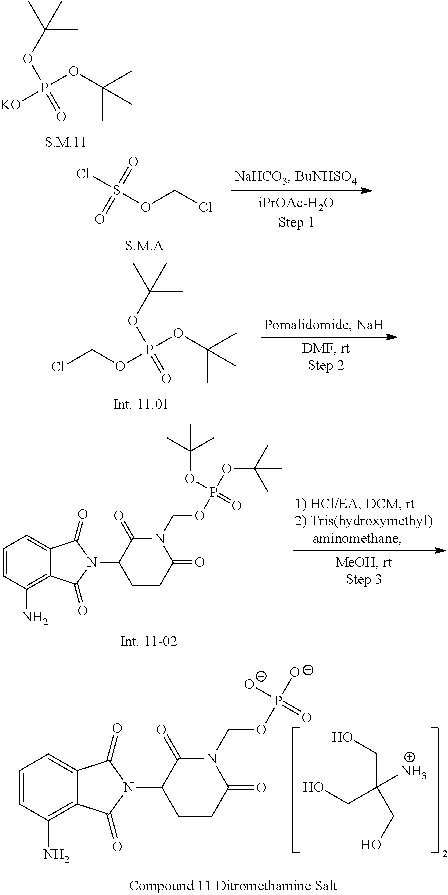

Step 1: S.M.11 (2.50 g, 10 mmol, 1.00 eq), sodium bicarbonate (3.78 g, 45 mmol, 4.50 eq), tetrabutylammonium hydrogen sulfate (169 mg, 0.5 mmol, 0.05 eq), water (12 mL), and isopropyl acetate (14 mL) were added to a 100 mL 3-neck reaction flask; and after stirring for 30 min, S.M.A (2.97 g, 18 mmol, 1.80 eq) was added dropwise and the reaction was stirred for about 13 h. The reaction was stopped and subjected to liquid separation, and the organic phase was concentrated under reduced pressure to give 2.20 g of a colorless liquid with a yield of 85.2%.

Step 2: Under nitrogen atmosphere protection, pomalidomide (1.00 g, 3.66 mmol, 1.00 eq) and DMF (10 mL) were added to a 100 mL three-mouth reaction flask; after stirring for 1 h, sodium hydride (60%) (175 mg, 4.39 mmol, 1.20 eq) was added and stirring was continued for 40 min; and then Int. 11.01 (0.95 g, 3.66 mmol, 1.00 eq) was added and stirring was continued for 27 h. The reaction was stopped, the system was poured into a 5% ammonium chloride (200 mL) solution, and the solid was precipitated with stirring, the solid was filtered, and the filter cake was dissolved in 200 mL of DCM, concentrated under reduced pressure and purified by column chromatography (PE:EA=1.5:1) to give 650 mg of a yellow solid product with a yield of 35.9%.

Step 3: Under nitrogen protection, Int11. 02 (490 mg, 1 mmol, 1.00 eq) and DCM (20 mL) were added to a 100 mL three-mouth reaction flask; and after stirring at room temperature for 10 min, HCl/EA (20 mL, 20 mmol, 20.0 eq) was added and stirring was continued for 45 min. The reaction was stopped, and the hydrochloric acid in the reaction system was vacuumed for 20 minutes with a water pump, and was then subjected to suction filtration under a nitrogen atmosphere to give a yellow solid product which was directly dissolved in 10 mL of methanol and used as raw material for the next step. Undernitrogen protection, a methanol solution of the above intermediate was added to a 100 mL three-mouth reaction flask, tributylamine alcohol (243 mg, 2 mmol, 2.00 eq) in 20 mL of methanol was added dropwise with stirring at room temperature, and was stirred for 10 min, 25 mL of acetone was added dropwise to the system, and a large amount of solid was precipitated in the system and stirred for 40 minutes. A filter cake was obtained by filtration, and was vacuum-dried to give 170 mg of a yellow solid product with a yield of 27.2%.

HPLC:99.73

LCMS:382(M-H-2tris)

¹H NMR (400 MHz, DMSO), δ 7.52-7.48 (t, 1H), 7.05-7.03 (t, 2H), 5.92 (m, 2H), 5.16-5.11 (dd, 1H), 3.37 (s, 12H), 3.03-2.95 (m, 1H), 2.82-2.78 (d, 1H), 2.64-2.53 (m, 1H), 2.10-2.03 (m, 1H)

### EXAMPLE 12

### Determination of Solubility of the Compound of the Present Invention

1 mg of compound 1 was mixed with 1 mL of water, stirred at room temperature, and completely dissolved, and the supernatant was taken as a test sample solution. The solubility of the test sample solution was determined by HPLC, and the results were as shown in Table 1.

50 mg of the hydrochloride or tromethamine salt of the compound was mixed with 150 μl of water, stirred at room temperature, and completely dissolved, and the supernatant was taken as a test sample solution. The solubility of the test sample solution was determined by HPLC, and the results were as shown in Table 1.

TABLE 1

| | Minimum solubility in |
|---|---|
| | water at room temperature |
| Sample | (mg/ml) |
| Compound 1 | 1 |
| Hydrochloride salt of compound 1 | >300 |
| Hydrochloride salt of compound 2 | >300 |
| Hydrochloride salt of compound 3 | >300 |
| Hydrochloride salt of compound 4 | >300 |
| Hydrochloride salt of compound 5 | >300 |
| Hydrochloride salt of compound 6 | >300 |
| Hydrochloride salt of compound 7 | >300 |
| Hydrochloride salt of compound 10 | >300 |
| Ditromethamine salt of compound 11 | >300 |

In this test, the saturated solubility of the sample is not determined, but an appropriate amount of the sample is selected to determine the solubility of the sample. It can be seen from the test that the compound (1 mg) or the salt (300 mg) of the present invention can be completely dissolved in 1 mL of water, the solubility of the compound is not less than 1 mg/ml, and the salt solubility is not less than 300 mg/ml. However, the solubility of pomalidomide in water is about 0.01 mg/ml, which is much lower than the solubility of the compound of the present invention or the salt thereof.

### EXAMPLE 13

### Determination of Stability of the Compound of the Present Invention

An appropriate amount of the sample was independently placed under different environmental conditions, and the percentage of the sample at day 0, 5, 10 or 30 was determined to evaluate the stability of the sample.

TABLE 2

| | Hydrochloride | Hydrochloride | Ditromethamine |
|---|---|---|---|
| | salt of | salt of | salt of |
| Sample | Compound 1 | Compound 2 | Compound 11 |
| Day 0 | 97.1% | 96.1% | 99.9% |
| Day 5/RT/75% | / | / | 79.8% |
| RH/Dark | | | |
| Day 5/RT/92.5% | / | / | 78.4% |
| RH/Dark | | | |
| Day 5/40° C./60% | / | / | 92.8% |
| RH/Dark | | | |
| Day 5/60° C./75% | / | / | 15.6% |
| RH/Dark | | | |
| Day 5/Light | / | / | 99.5% |
| Day 10/RT/75% | 96.8% | / | / |
| RH/Dark | | | |
| Day 10/RT/92.5% | 95.5% | 93.3% | / |
| RH/Dark | | | |
| Day 10/40° C./60% | 96.9% | 43.5% | / |
| RH/Dark | | | |
| Day 10/60° C./75% | 96.4% | 13.3% | / |
| RH/Dark | | | |
| Day 10/Light | 96.7% | / | / |
| Day 30/RT/75% | 96.1% | / | / |
| RH/Dark | | | |
| Day 30/RT/92.5% | 93.3% | / | / |
| RH/Dark | | | |
| Day 30/40° C./60% | 95.1% | / | / |
| RH/Dark | | | |
| Day 30/60° C./75% | 94.7% | / | / |
| RH/Dark | | | |
| Day 30/Light | 95.0% | / | / |

Note:
RT = room temperature,
RH = relative humidity

### Example 14

### Pharmacokinetic Data

In the present embodiment, male SD rats (SPF grade), which weigh 250 g-330 g, and which were purchased from Beijing Vital River Laboratory Animal Technology Co., Ltd., were older than 8 weeks, and were housed in groups in a polycarbonate cage filled with padding (up to 5 animals/gender/cage). These rats were free to drink water and eat qualified feed 5CC4 (the same as 5CR4, PMI Nutrition International LLC, USA) daily.

Adopting grouping of random grouping design, the SD rats were divided into the hydrochloride salt of compound 1 group, the ditromethamine salt of compound 11 group, and the pomalidomide group. Rats of each group were administered by intragastrically (i.g.) with the doseset at 2 mg/kg (based on pomalidomide).

Test method: Blood samples were taken at 0.25, 0.5, 1, 2, 3, 4, 6, 8, 12, 24, 36 and 48 hours after administration and before administration, whole blood was collected and placed into a pre-labeled EDTA-2K anticoagulation tube and stored on wet ice, within 30 minutes, followed by centrifugation at 4° C., 3500 rpm for 5 minutes. Plasma samples were immediately stored in dry ice and then transferred to a −60° C. to −80° C. refrigerator. The hydrochloride salt of compound 1, the ditromethamine salt of compound 11, and the pomalidomide in the plasma were analyzed and detected by LC/MS/MS. The pharmacokinetic parameters of the rats after administration were calculated using the non-compartmental model of the WinNonlin software.

Results: Each group of rats, after administration of pomalidomide (2 mg/kg) or equal molar of various compounds, the pharmacokinetic parameters of the pomalidomide concentration in plasma over time are as shown in the table below.

TABLE 3

| | Hydrochloride | Ditromethamine | |
|---|---|---|---|
| | salt of | salt of | |
| Compound | Compound 1 | Compound 11 | Pomalidomide |
| Dosage (mg/kg) | 2.95 | 2.8 | 2 |
| Tₘₐₓ (h) | 0.58 ± 0.38 | 0.33 ± 0.14 | 2.33 ± 0.58 |
| Cₘₐₓ (ng/mL) | 1100 ± 214 | 1240 ± 383 | 366 ± 62 |
| t_{1/2} (h) | 2.69 ± 0.53 | 2.09 ± 0.27 | 2.62 ± 0.28 |
| AUCₗₐₛₜ (h*ng/mL) | 4660 ± 249 | 4510 ± 1420 | 2570 ± 383 |

Conclusions: It can be seen from the above test results that after the administration of each group, only pomalidomide is detected in vivo, indicating that all the compounds are rapidly metabolized to pomalidomide after entering the body. At a dose of 2 mg/kg, after administration, compared with pomalidomide, the Cₘₐₓ value of the compound of the present invention increases by more than three times, and AUCₗₐₛₜ increases by more than two times. The Cₘₐₓ of compound 1 and compound 11 indicates that the compound of the present invention has a better oral bioavailability compared to pomalidomide.

1. A compound of formula (I) or a pharmaceutically acceptable salt thereof, 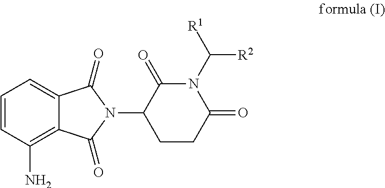
wherein:
R¹ is selected from H, substituted or unsubstituted C₁₋₆ alkyl, substituted or unsubstituted C₁₋₆ alkoxy, substituted or unsubstituted C₂₋₆ alkenyl, or substituted or unsubstituted C₂₋₆ alkynyl, wherein the aforementioned substituted substituent is selected from C₁₋₆ alkyl or C₁₋₆ alkoxy;
R² is selected from H, —OR³, —SR³, —NHR³, substituted or unsubstituted C₃₋₁₀ heterocyclyl, substituted or unsubstituted C₃₋₁₀ heterocyclic aryl, or substituted or unsubstituted C₃₋₁₀ cycloalkyl, wherein the aforementioned substituted substituent is selected from C₁₋₆ alkyl, C₁₋₆ alkoxy, carbonyl, carboxyl, amino, or hydroxy;
R³ is selected from —C(O)(CH)(R⁴)(R⁵), —P(O)(OR⁶)(OR⁷), —P(O)₂(OR⁶)M, or —P(O)₃MY;
R⁴ is selected from hydrogen, amino, hydroxy, halogen, or C₁₋₆ alkyl;
R⁵ is selected from hydrogen, substituted or unsubstituted C₁₋₁₆ alkyl, substituted or unsubstituted C₁₋₆ alkoxy, substituted or unsubstituted C₃₋₁₀ heterocyclyl, substituted or unsubstituted C₃₋₁₀ heterocyclic aryl, substituted or unsubstituted C₃₋₁₀ cycloalkyl, phenyl, benzyl, —(CH₂)nSCH₃, —(CH₂)mNHCH₃, or —(CH₂)mN(CH₃)₂, wherein the aforementioned substituted substituent is selected from amino, hydroxyl, carboxyl, —SH, —C(O)NH₂, C₁₋₆ alkyl;
R⁶ and R⁷ are each independently selected from hydrogen or C₁₋₆ alkyl; M and Y are each independently selected from a monovalent cation, or MY is a divalent cation; and
m and n are each independently selected from 1, 2, 3, 4, 5 or 6.

2. The compound of claim 1 or a pharmaceutically acceptable salt thereof, wherein R¹ is selected from H, substituted or unsubstituted methyl, substituted or unsubstituted ethyl, substituted or unsubstituted propyl, substituted or unsubstituted butyl, substituted or unsubstituted pentyl, substituted or unsubstituted hexyl, wherein the aforementioned substituted substituent is selected from methyl, ethyl, or propyl.

3. The compound of claim 1 or a pharmaceutically acceptable salt thereof, wherein R² is selected from H, —OR³, substituted or unsubstituted tetrahydrofuranyl, substituted or unsubstituted tetrahydropyrrolyl, substituted or unsubstituted furanyl, substituted or unsubstituted thienyl, substituted or unsubstituted pyrrolyl, substituted or unsubstituted imidazolyl, substituted or unsubstituted pyrazolyl, substituted or unsubstituted thiazolyl, substituted or unsubstituted oxazolyl, substituted or unsubstituted pyridyl, substituted or unsubstituted pyrimidyl, substituted or unsubstituted pyridazinyl, substituted or unsubstituted pyrazinyl, substituted or unsubstituted purinyl, substituted or unsubstituted quinolyl, substituted or unsubstituted isoquinolyl, substituted or unsubstituted indolyl, substituted or unsubstituted cyclopropyl, substituted or unsubstituted cyclohexyl, substituted or unsubstituted cyclobutyl, and substituted or unsubstituted cyclopentyl, 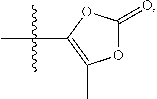 [SMILES: *c1oc(=O)oc1C]
wherein the aforementioned substituted substituent is selected from methyl, ethyl, propyl, carbonyl, carboxyl, amino, or hydroxy.

4. The compound of claim 3 or a pharmaceutically acceptable salt thereof, wherein R³ is selected from —C(O)(CH)(R⁴)(R⁵), wherein:
R⁴ is selected from hydrogen, amino, methyl, ethyl, or propyl; and
R⁵ is selected from hydrogen, substituted or unsubstituted tetrahydrofuranyl, substituted or unsubstituted tetrahydropyrrolyl, substituted or unsubstituted furanyl, substituted or unsubstituted thienyl, substituted or unsubstituted pyrrolyl, substituted or unsubstituted imidazolyl, substituted or unsubstituted pyrazolyl, substituted or unsubstituted thiazolyl, substituted or unsubstituted oxazolyl, substituted or unsubstituted pyridyl, substituted or unsubstituted pyrimidyl, substituted or unsubstituted pyridazinyl, substituted or unsubstituted pyrazinyl, substituted or unsubstituted purinyl, substituted or unsubstituted quinolyl, substituted or unsubstituted isoquinolyl, substituted or unsubstituted indolyl, substituted or unsubstituted cyclopropyl, substituted or unsubstituted cyclohexyl, substituted or unsubstituted cyclobutyl, substituted or unsubstituted cyclopentyl, phenyl, benzyl, —(CH₂)nSCH₃, —(CH₂)mNHCH₃, —(CH₂)mN(CH₃)₂, 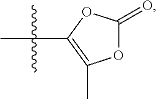 [SMILES: *c1oc(=O)oc1C]
wherein the aforementioned substituted substituent is selected from amino, hydroxy, carboxyl, —SH, —C(O)NH₂, methyl, ethyl, or propyl.

5. The compound of claim 4 or a pharmaceutically acceptable salt thereof, wherein n is selected from 1, 2 or 3, and m is selected from 1, 2, 3, 4 or 5.

6. The compound of claim 1 or a pharmaceutically acceptable salt thereof, wherein R³ is selected from —P(O)(OR⁶)(OR⁷), —P(O)₂(OR⁶)M, —P(O)₃MY, wherein R⁶ and R⁷ are each independently selected from hydrogen, methyl, ethyl, propyl, butyl, pentyl, or hexyl, M and Y are each independently selected from sodium ions, potassium ions, or MY is a divalent cation selected from calcium ions, magnesium ions.

7. The compound of claim 1 or a pharmaceutically acceptable salt thereof, wherein:
R¹ is selected from H, methyl, ethyl or propyl;
R² is selected from H, —OR³, substituted or unsubstituted furanyl, substituted or unsubstituted thienyl, substituted or unsubstituted pyrrolyl, substituted or unsubstituted imidazolyl, substituted or unsubstituted pyrazolyl, substituted or unsubstituted thiazolyl, substituted or unsubstituted oxazolyl, substituted or unsubstituted pyridyl, substituted or unsubstituted pyrimidyl, substituted or unsubstituted pyridazinyl, substituted or unsubstituted pyrazinyl, substituted or unsubstituted purinyl, substituted or unsubstituted quinolyl, substituted or unsubstituted isoquinolyl, substituted or unsubstituted indolyl, 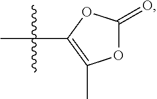 [SMILES: *c1oc(=O)oc1C]
wherein the aforementioned substituted substituent is selected from methyl, ethyl, or propyl;
R³ is selected from —C(O)(CH)(R⁴)(R⁵), —P(O)(OR⁶)(OR⁷), —P(O)₂(OR⁶)M, or —P(O)₃MY;
R⁴ is selected from hydrogen, amino, methyl, ethyl, or propyl;
R⁵ is selected from hydrogen, methyl, ethyl, propyl, butyl, pentyl, hexyl, heptyl, octyl, nonyl, decyl, tetrahydrofuranyl, tetrahydropyrrolyl, furanyl, thienyl, pyrrolyl, imidazolyl, pyrazolyl, thiazolyl, oxazolyl, pyridyl, pyrimidyl, pyridazinyl, pyrazinyl, purinyl, quinolyl, isoquinolyl, indolyl, phenyl, benzyl, —(CH₂)nSCH₃, —(CH₂)mNHCH₃, or —(CH₂)mN(CH₃)₂;
R⁶ and R⁷ are each independently selected from hydrogen, methyl, ethyl, and propyl; M and Y are each independently selected from sodium ions, potassium ions, or MY is a divalent cation selected from calcium ions,magnesium ions; and
m and n are each independently selected from 1, 2, 3, 4, 5 or 6.

8. The compound of claim 7 and a stereoisomer or a pharmaceutically acceptable salt thereof, wherein,
R¹ is selected from H and methyl;
R² is selected from H, —OR³, 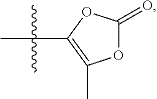 [SMILES: *c1oc(=O)oc1C]
wherein the substituent is selected from methyl, ethyl, or propyl;
R³ is selected from —C(O)(CH)(R⁴)(R⁵), —P(O)(OR⁶)(OR⁷), —P(O)₂(OR⁶)M, or —P(O)₃MY;
R⁴ is selected from hydrogen or amino;
R⁵ is selected from hydrogen, methyl, ethyl, propyl, butyl, pentyl, hexyl, heptyl, octyl, nonyl, decyl, tetrahydrofuranyl, tetrahydropyrrolyl, furanyl, thienyl, pyrrolyl, imidazolyl, pyrazolyl, thiazolyl, oxazolyl, pyridyl, pyrimidyl, pyridazinyl, pyrazinyl, purinyl, quinolyl, isoquinolyl, indolyl, phenyl, benzyl, —(CH₂)nSCH₃, —(CH₂)mNHCH₃, or —(CH₂)mN(CH₃)₂;
R⁶ and R⁷ are hydrogen; M and Y are each independently selected from sodium ions, potassium ions, or MY is a divalent cation selected from calcium ions, magnesium ions; and
m and n are each independently selected from 1, 2, 3, 4, 5 or 6.

9. The compound of claim 1 or a pharmaceutically acceptable salt thereof, wherein the compound has one of the following structures:

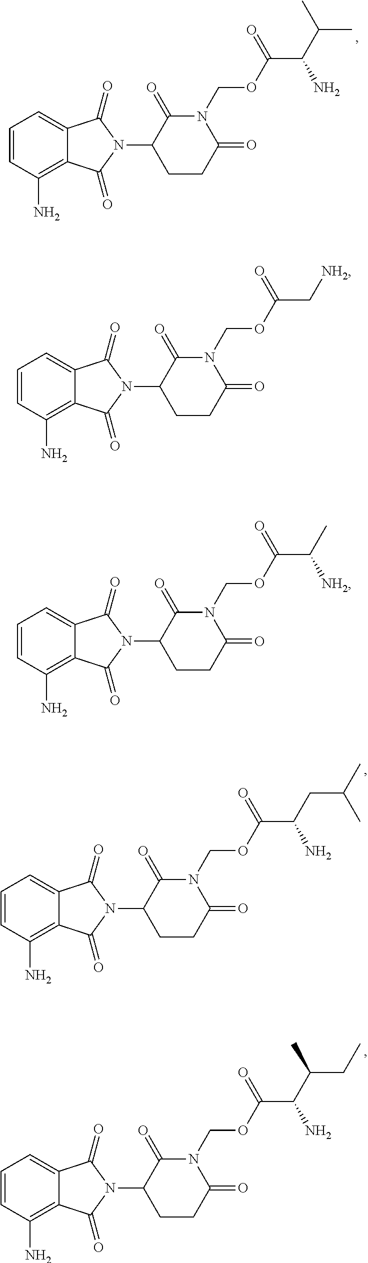 [SMILES: CC(C)[C@H](N)C(=O)OCN1C(=O)CCC(N2C(=O)c3cccc(N)c3C2=O)C1=O ; NCC(=O)OCN1C(=O)CCC(N2C(=O)c3cccc(N)c3C2=O)C1=O ; C[C@H](N)C(=O)OCN1C(=O)CCC(N2C(=O)c3cccc(N)c3C2=O)C1=O ; CC(C)C[C@H](N)C(=O)OCN1C(=O)CCC(N2C(=O)c3cccc(N)c3C2=O)C1=O ; CC[C@H](C)[C@H](N)C(=O)OCN1C(=O)CCC(N2C(=O)c3cccc(N)c3C2=O)C1=O]

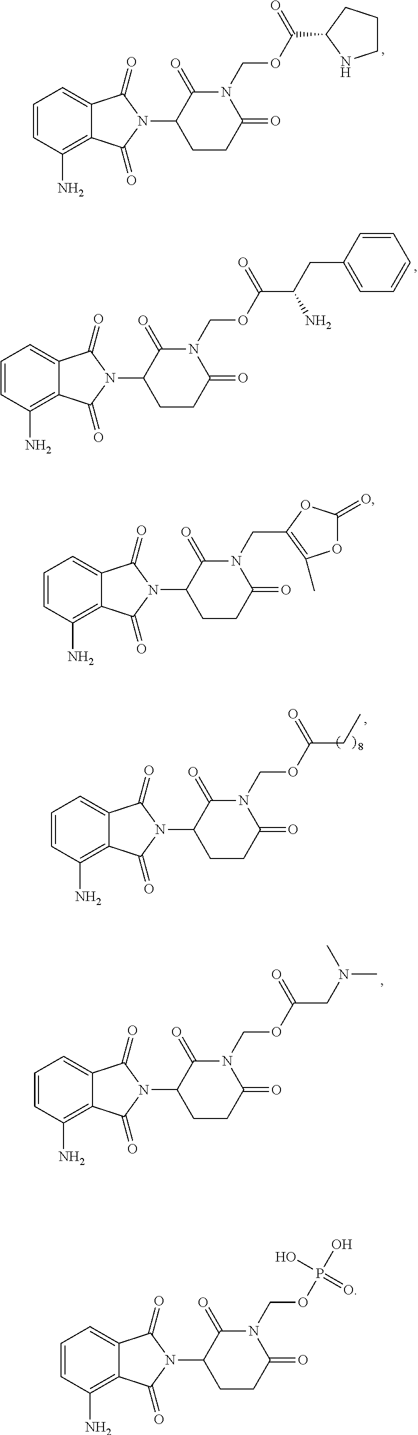

10. The compound of claim 1 or a pharmaceutically acceptable salt thereof, wherein the pharmaceutically acceptable salt is a hydrochloride or tromethamine salt.

11. A pharmaceutical composition comprising a therapeutically effective amount of the compound of claim 1 or a pharmaceutically acceptable salt thereof, and a pharmaceutically acceptable carrier and/or excipient.

12. A method of treating a cancer comprising: administering a compound according to claim 1 or a pharmaceutically acceptable salt thereof to said patient.

13. The method of claim 12, wherein the cancer is multiple myeloma, prostate cancer.
